(19) United States
(12) Patent Application Publication
(10) Pub. No.: US 2026/0256442 A1
(43) Pub. Date: Sep. 3, 2026

(54) RADIOGRAPHY APPARATUS, METHOD OF OPERATING RADIOGRAPHY APPARATUS, AND PROGRAM OF OPERATING RADIOGRAPHY APPARATUS

(71) Applicant: FUJIFILM CORPORATION, Tokyo (JP)

(72) Inventors: Hidenori MATSUDA, Kanagawa (JP); Hisatsugu HORIUCHI, Kanagawa (JP); Takeyasu KOBAYASHI, Kanagawa (JP)

(21) Appl. No.: 19/537,590

(22) Filed: Feb. 12, 2026

(30) Foreign Application Priority Data
Feb. 28, 2025 (JP) 2025-032193

(51) Int. Cl. A61B 6/00

(52) U.S. Cl. CPC A61B 6/547; A61B 6/545

(57) ABSTRACT

A radiography apparatus is a radiography apparatus used for radiography, the radiography apparatus comprising: a radiation source or a radiographic image detection device; a driving mechanism that is autonomously drivable; and a processor that executes driving control related to the driving mechanism, in which the processor switches between a first mode in which a load on the processor is relatively large and a second mode in which the load is relatively small, according to a preset condition, with respect to a mode in which the driving control is executed.

### CROSS-REFERENCE TO RELATED APPLICATIONS

The present application claims priority under 35 U.S.C. §119 to Japanese Patent Application No. 2025-032193, filed on February 28, 2025. The above application is hereby expressly incorporated by reference, in its entirety, into the present application.

### BACKGROUND

### 1. Technical Field

The technology of the present disclosure relates to a radiography apparatus, a method of operating the radiography apparatus, and a program of operating the radiography apparatus.

### 2. Description of the Related Art

In a medical field, a radiography apparatus including a driving mechanism is known. As such a radiography apparatus, for example, a radiation generation apparatus is known in which a body part including a radiation source that emits radiation toward a subject is mounted on a carriage unit including wheels. For example, the radiation generation apparatus is used for so-called ward round imaging in which a patient as a subject is imaged while moving around a ward.

WO2017/043040A discloses a radiation generation apparatus that is manually drivable and includes a camera that images a surrounding environment. In WO2017/043040A, the manual driving to a target position such as a side of a decubitus imaging table (bed) on which an electronic cassette is installed is assisted by notifying an operator such as a radiologic technologist of a path to avoid an obstacle captured by the camera.

### SUMMARY

The inventors have considered adopting a SLAM method in which autonomous driving is performed to a target position using, for example, a simultaneous localization and mapping (SLAM) technology in such a driving type radiography apparatus. In a case in which the autonomous driving can be performed, it is possible to further reduce a burden on the operator.

The SLAM method is a technology of creating a map by recognizing a surrounding environment and estimating a self-position based on the map in parallel, and a camera is used as an environment information sensor that acquires surrounding environment information. A processor that executes driving control related to the driving mechanism repeats updating a map of a surrounding environment and estimating a self-position on the map based on an image acquired by the camera.

The processor of the radiography apparatus may execute imaging control related to imaging in addition to such driving control. Therefore, in a case in which a load on the processor is large in the driving control, there is a concern that resources of the processor to be assigned to other controls such as the imaging control are insufficient.

The disclosed technology provides a radiography apparatus, an operation method of a radiography apparatus, and an operation program of a radiography apparatus that can appropriately allocate resources of a processor in a case of executing driving control related to a drivable driving mechanism, as compared to the related art.

A radiography apparatus according to the present disclosed technology is a radiography apparatus which is used for radiography, the radiography apparatus comprising: a radiation source or a radiographic image detection device; a driving mechanism that is autonomously drivable; and a processor configured to execute driving control related to the driving mechanism, in which the processor switches between a first mode in which a load on the processor is relatively large and a second mode in which the load is relatively small, according to a preset condition, with respect to a mode in which the driving control is executed.

The processor may further execute alignment control that supports relative alignment between the radiation source and the radiographic image detection device and/or a subject, and imaging control related to the radiation source or the radiographic image detection device. The preset condition may include start of at least one of the alignment control or the imaging control. The processor may execute the driving control in the first mode before the start of at least one of the controls and may execute the driving control in the second mode in a case of starting at least one of the controls.

The first mode may be a mode in which autonomous driving is performed toward a preset target position by executing self-position estimation based on surrounding environment information. The second mode may be a mode in which autonomous driving is performed toward a preset target position by detecting a movement amount of the driving mechanism and executing self-position estimation based on the detected movement amount.

The first mode and the second mode may be modes in which autonomous driving is performed toward a preset target position by executing self-position estimation based on surrounding environment information, and the first mode and the second mode may have different operation conditions.

The operation condition may include at least one of an acquisition frequency of the surrounding environment information, a moving speed of the driving mechanism, an information amount of the environment information, or a calculation amount of the processor. The second mode may be a mode that satisfies at least one of a condition in which the acquisition frequency is low, a condition in which the moving speed is slow, a condition in which the information amount of the environment information is small, or a condition in which the calculation amount is small, as compared with the first mode.

The first mode may be a mode in which autonomous driving is performed toward a preset target position by executing self-position estimation based on surrounding environment information, and the preset condition may include that the self-position has entered a preset proximity range of the target position. The processor may execute the driving control in the first mode before entering the proximity range and execute the driving control in the second mode after entering the proximity range.

The radiography apparatus according to the present disclosed technology may be driven by a battery.

The preset condition may include that a remaining amount of the battery has decreased to be less than a preset threshold value. The processor may execute the driving control in the first mode in a case where the remaining amount is equal to or larger than the threshold value and execute the driving control in the second mode in a case where the remaining amount has decreased to be less than the threshold value.

The radiography apparatus according to the present disclosed technology may include the radiation source and may be a radiation generation apparatus.

An operation method of a radiography apparatus according to the present disclosed technology is an operation method of a radiography apparatus which is used for radiography, and which includes a radiation source or a radiographic image detection device, a driving mechanism that is autonomously drivable, and a processor configured to execute driving control related to the driving mechanism, the operation method comprising: switching, by the processor, between a first mode in which a load on the processor is relatively large and a second mode in which the load is relatively small, according to a preset condition, with respect to a mode in which the driving control is executed.

An operation program of a radiography apparatus according to the present disclosed technology is an operation program of a radiography apparatus which is used for radiography, and which includes a radiation source or a radiographic image detection device, a driving mechanism that is autonomously drivable, and a processor configured to execute driving control related to the driving mechanism, the operation program causing the processor to execute a process comprising: switching between a first mode in which a load on the processor is relatively large and a second mode in which the load is relatively small, according to a preset condition, with respect to a mode in which the driving control is executed.

According to the disclosed technology, it is possible to appropriately allocate resources of a processor in a case of executing driving control related to a drivable driving mechanism, as compared to the related art.

### BRIEF DESCRIPTION OF THE DRAWINGS

FIG. 1 is a diagram showing a state of decubitus imaging using a radiography system.

FIG. 2 is a diagram showing a state of upright imaging using the radiography system.

FIG. 3 is a diagram showing a radiation generation apparatus in which a radiation source is in an accommodation position.

FIG. 4 is a block diagram showing an electric configuration of the radiation generation apparatus.

FIG. 5 is a diagram showing a state in which the radiation generation apparatus drives from a standby position toward a first target position in the decubitus imaging and stops at the first target position.

FIG. 6 is a diagram showing a state in which the radiation generation apparatus drives from the standby position toward a second target position in the upright imaging and stops at the second target position.

FIG. 7 is a diagram showing processing of each processing unit of a processor related to the driving control.

FIG. 8 is a diagram showing processing of each processing unit of the processor related to the driving control following FIG. 7.

FIG. 9 is a diagram showing processing of each processing unit of a processor related to the alignment control.

FIG. 10 is a diagram showing processing in a learning phase of a cassette contour extraction model.

FIG. 11 is a diagram showing a state of the alignment control.

FIG. 12 is a diagram showing an alignment support screen.

FIG. 13 is a diagram showing contents of the imaging control.

FIG. 14 is a conceptual diagram of switching between the first mode and the second mode in the driving control.

FIG. 15 is a flowchart showing an example of switching the mode based on the start of the alignment control.

FIG. 16 is a diagram showing an operation example of the radiography apparatus in a case of FIG. 15.

FIG. 17 is a flowchart of Modification Example 1.

FIG. 18 is a flowchart of Modification Example 2.

FIG. 19 is a diagram showing an example in which both the first mode and the second mode are the SLAM method.

FIG. 20 is a flowchart showing an example of switching the mode according to the remaining amount of the battery.

### DETAILED DESCRIPTION

### First Embodiment

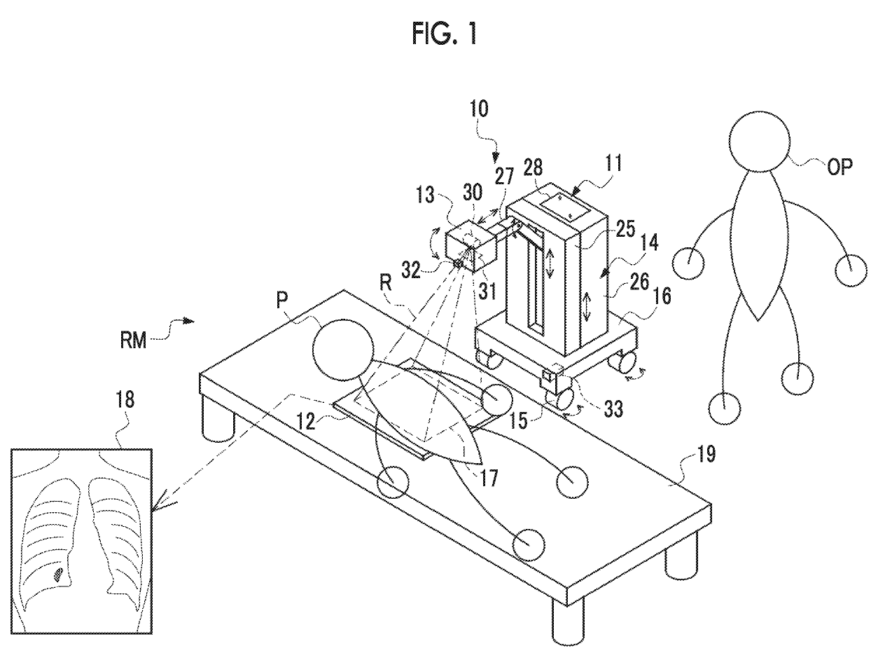

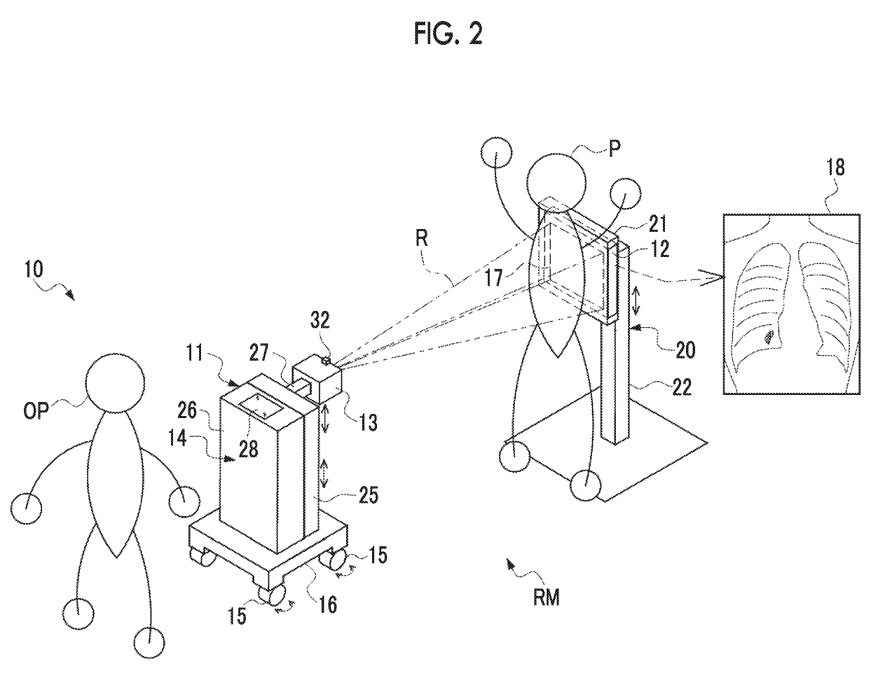

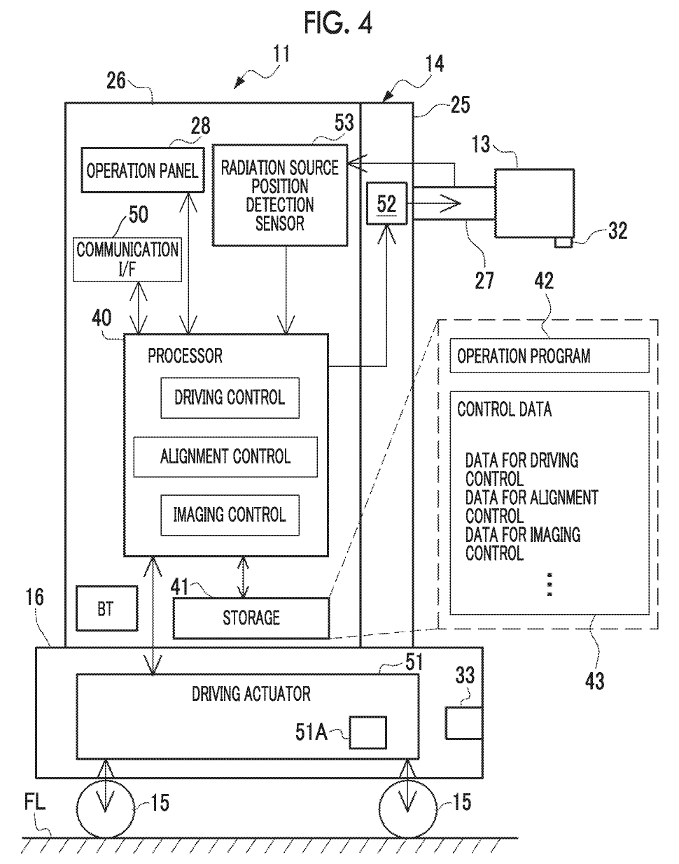

As shown in FIGS. 1 and 2 as an example, a radiography system 10 comprises a radiation generation apparatus 11 and an electronic cassette 12. The radiation generation apparatus 11 has a configuration in which a body part 14 including a radiation source 13 that emits radiation R toward a patient P, which is an example of a subject, is mounted on a carriage unit 16 including wheels 15. The radiation R is, for example, X-rays. A battery BT (see FIG. 4) is mounted in the radiation generation apparatus 11, and the radiation generation apparatus 11 can be driven by being supplied with power from the battery BT. In addition, the radiation generation apparatus 11 can also be driven while being supplied with power from a commercial power supply through a power supply cord. The radiation generation apparatus 11 is a device used for radiography and is an example of a "radiography apparatus" according to the disclosed technology. The carriage unit 16 is an example of a "driving mechanism" according to the disclosed technology. Further, the electronic cassette 12 is an example of a “radiographic image detection device” according to the technology of the present disclosure.

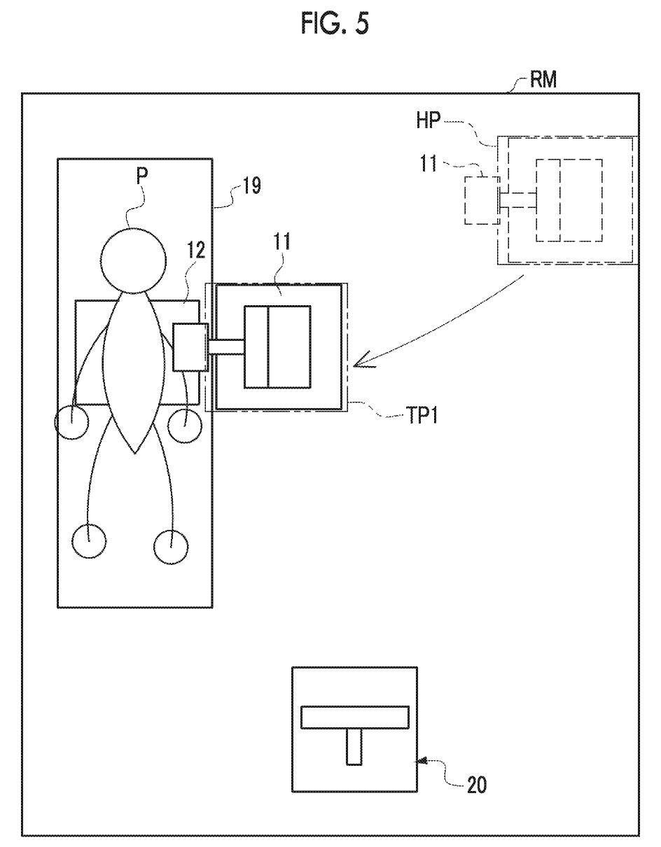

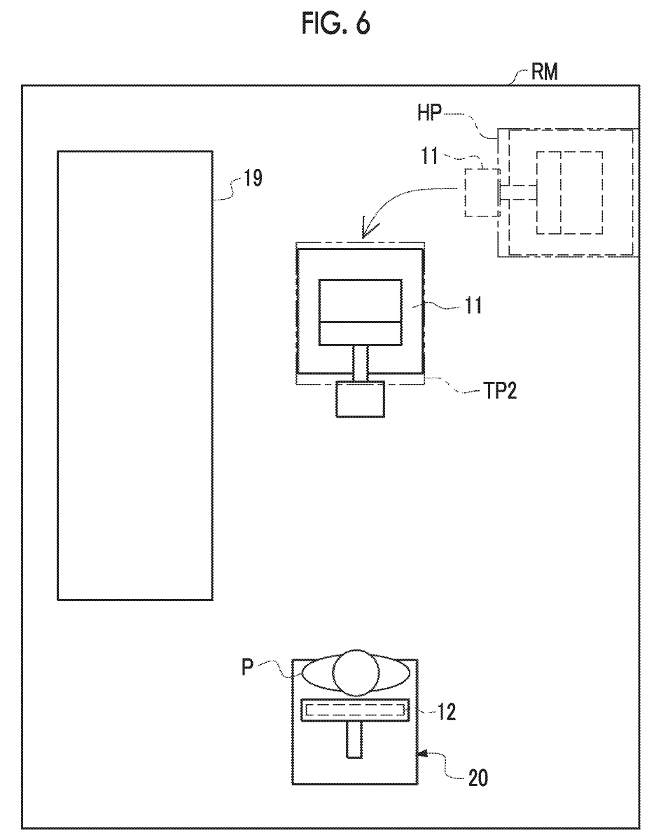

The radiation generation apparatus 11 can be moved in an imaging room RM (see also FIGS. 5 and 6). In addition, the radiation generation apparatus 11 is used for so-called ward round imaging in which a patient P is imaged while moving around a ward. Therefore, the radiation generation apparatus 11 is also called a ward round cart. Alternatively, the radiation generation apparatus 11 is also used for imaging in an emergency room. In addition, the radiation generation apparatus 11 can also be carried into an operating room and used during surgery.

The electronic cassette 12 has a configuration in which a sensor panel as a radiation detector that detects the radiation R is built in a portable housing. The electronic cassette 12 is driven by the battery BT and performs wireless communication with the radiation generation apparatus 11. The sensor panel has a detection surface 17 in which a plurality of pixels that generate signal charges in response to the radiation R or visible light converted from the radiation R by a scintillator are arranged in a matrix. The electronic cassette 12 detects the radiation R emitted from the radiation source 13 and transmitted through the patient P, and outputs a radiographic image 18 of the patient P.

Since the electronic cassette 12 is portable and wireless, as shown in FIG. 1, the electronic cassette 12 can be used for so-called free imaging in which the electronic cassette 12 is installed on a decubitus imaging table (bed) 19 installed in the imaging room RM (under the patient P) to perform radiography. More specifically, FIG. 1 shows a state in which the electronic cassette 12 is inserted between the decubitus imaging table 19 and the patient P to perform imaging for imaging the patient P lying on the decubitus imaging table 19 (see also FIG. 5). In addition, as shown in FIG. 2, the electronic cassette 12 can also be used by being accommodated in a holder 21 of an upright imaging table 20 installed in the imaging room RM (see also FIG. 6). The holder 21 can be raised and lowered in the up-down direction with respect to a support column 22.

The body part 14 has a rectangular-parallelepiped shape and is erected at the center of the carriage unit 16. The body part 14 is divided into a movable part 25 on a front side and a fixing unit 26 on a rear side. The movable part 25 can be raised and lowered in the up-down direction with respect to the fixing unit 26. The fixing unit 26 is fixed to the carriage unit 16.

A base end of an arm 27 is attached to the movable part 25. More specifically, the arm 27 is divided into a first portion in which the base end is attached to the movable part 25 and a second portion in which the base end is attached to the first portion. The radiation source 13 is attached to a distal end of the second portion, which is a free end opposite to the base end.

The first portion can be raised and lowered in the up-down direction with respect to the movable part 25 and can be rotated with respect to the movable part 25. The second portion can be bent in the up-down direction with respect to the first portion. Further, the second portion is extensible. The radiation source 13 can be rotated with respect to the second portion, that is, can be swung. By the displacement of the arm 27 and the displacement of the radiation source 13 with respect to the arm 27, a height position, a horizontal position, and a posture (orientation) of the radiation source 13 can be adjusted.

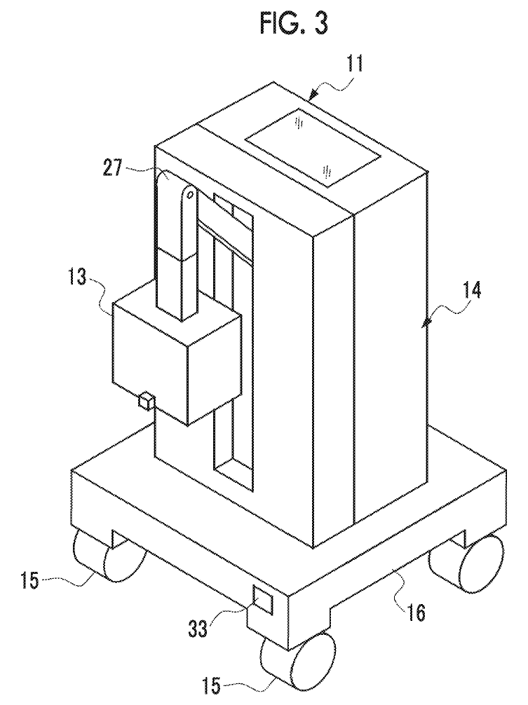

Since the second portion is bendable and extensible, the radiation source 13 can be moved to an imaging preparation position protruding toward the patient P and the electronic cassette 12 as shown in FIGS. 1 and 2 and an accommodation position pulled into the body part 14 as shown in FIG. 3 as an example. The imaging preparation position shown in FIGS. 1 and 2 is a position in a case in which the second portion is extended to a predetermined length, for example, a length of half of the longest length, and the second portion is bent with respect to the first portion such that the second portion is parallel to the horizontal direction. The accommodation position shown in FIG. 3 is a position in a case in which the second portion is shortened to the shortest and the second portion is folded as much as possible with respect to the first portion.

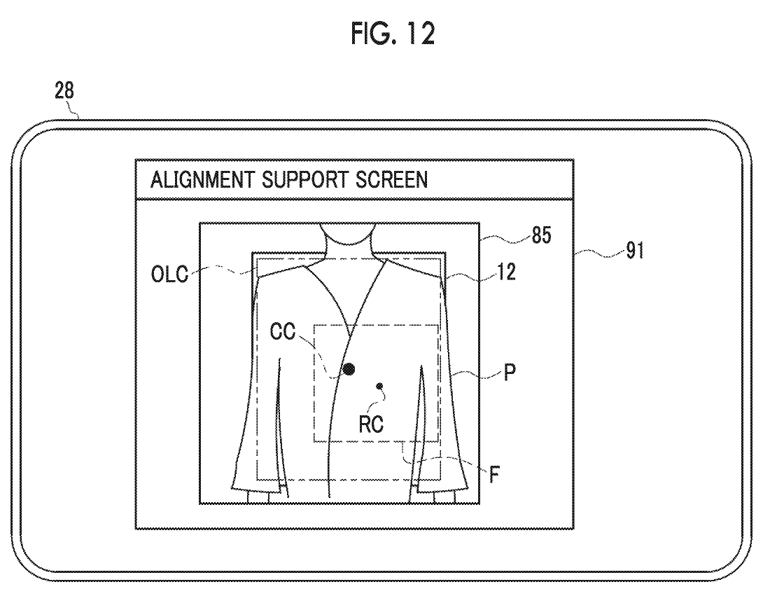

In FIGS. 1 and 2, an operation panel 28 is provided on an upper surface of the fixing unit 26. The operation panel 28 is configured by, for example, a touch panel display and has a function of displaying information in addition to an operation function. The operation panel 28 is operated by an operator OP such as a radiologic technologist. The operator OP sets an irradiation condition of the radiation R through the operation panel 28. In addition, the operator OP checks the radiographic image 18 through the operation panel 28. Further, as will be described later, the operator OP performs relative alignment (also referred to as positioning) between the radiation source 13 and the electronic cassette 12 with reference to an alignment support screen 91 (see FIG. 12) displayed on the operation panel 28.

In addition, an irradiation switch (not shown) is provided in the fixing unit 26. The irradiation switch is a switch that is provided to allow the operator OP to give an instruction to start irradiation of radiation. An extension cable is connected to the irradiation switch, and can be detached from the fixing unit 26 for use.

The radiation source 13 includes a radiation tube 30 and an irradiation field limiter 31. The radiation tube 30 generates the radiation R. The radiation tube 30 is provided with a filament, a target, a grid electrode, and the like (all are not illustrated). A tube voltage is applied between the filament that is a cathode and the target that is an anode from a voltage generator (not shown) built in the fixing unit 26. The filament releases thermal electrons according to the applied tube voltage toward the target. The target radiates the radiation R with collision of the thermoelectrons released from the filament. The grid electrode is disposed between the filament and the target, and changes a flow rate of the thermoelectrons from the filament toward the target in response to a voltage applied from the voltage generator. The flow rate of the thermal electrons from the filament toward the target is referred to as a tube current. The tube voltage and the tube current are set to the radiation source 13 as the irradiation condition with the irradiation time.

The irradiation field limiter 31 is also called a collimator or the like, and limits an irradiation field of the radiation R generated from the radiation tube 30. For example, the irradiation field limiter 31 has a configuration in which four shield plates formed of lead or the like shielding radiation R are disposed on respective sides of a quadrangle, and an emission opening of the quadrangle transmitting radiation is formed in a center portion. The irradiation field limiter 31 changes a size of the emission opening by changing a position of each shielding plate, thereby changing the irradiation field of the radiation R.

A camera 32 is attached to the radiation source 13. The camera 32 is used to support the alignment between the radiation source 13 and the electronic cassette 12. The camera 32 includes an imaging element that is sensitive to visible light, for example, a complementary metal oxide semiconductor (CMOS) image sensor or a charge-coupled device (CCD) image sensor. The camera 32 captures a video image. The camera 32 is an example of an "environment recognition sensor" according to the disclosed technology.

The wheels 15 are provided in four positions in front, behind, left, and right of the carriage unit 16. That is, the carriage unit 16 is a four-wheel type. Each wheel 15 is, for example, a revolution type that revolves around a revolution axis extending in a height direction (also referred to as a vertical direction) orthogonal to a rotation axis in a case of driving and rotating. The radiation generation apparatus 11 autonomously drives by the carriage unit 16.

Here, the autonomous driving refers to autonomously driving toward a set target position while recognizing a surrounding environment using the environment information sensor and estimating a self-position. For example, a position on a side of the decubitus imaging table 19 shown in FIG. 1 and a position facing the upright imaging table 20 shown in FIG. 2 are preset as the target position. The radiation generation apparatus 11 uses a SLAM method using a SLAM technology as one of driving control methods for realizing the autonomous driving.

A camera 33 is provided in the carriage unit 16. The camera 33 is used to support the autonomous driving of the body part 14. The camera 33 images a front side of the carriage unit 16. The camera 33 also includes an imaging element that is sensitive to visible light and captures a video image, as in the camera 32. The camera 33 is also an example of an "environment information sensor" according to the disclosed technology, as in the camera 32. The body part 14 and the radiation generation apparatus 11 can be manually driven by the operator OP in addition to the autonomous driving.

FIG. 4 is a block diagram showing an example of an electric configuration of the radiation generation apparatus 11. A processor 40 integrally controls the entire radiation generation apparatus 11. The processor 40 is configured by, for example, a central processing unit (CPU) and a memory such as a random access memory (RAM), and functions as various processing units by loading and executing various programs in the memory. Specifically, the processor 40 executes driving control, alignment control, and imaging control. The driving control is control related to the autonomous driving of the carriage unit 16. The alignment control is control related to the alignment between the radiation source 13 and the electronic cassette 12 as described above. The imaging control includes irradiation control of the radiation R by the radiation source 13 and output control of the radiographic image 18 by the electronic cassette 12.

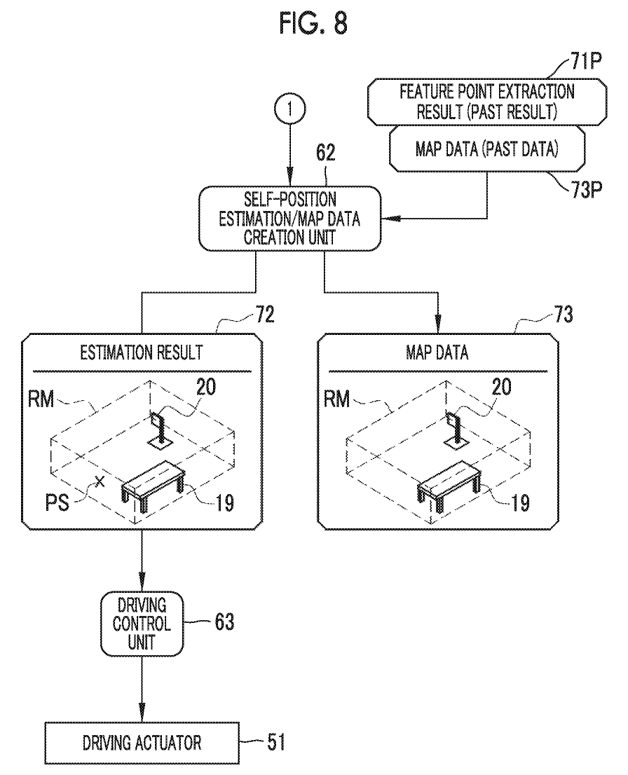

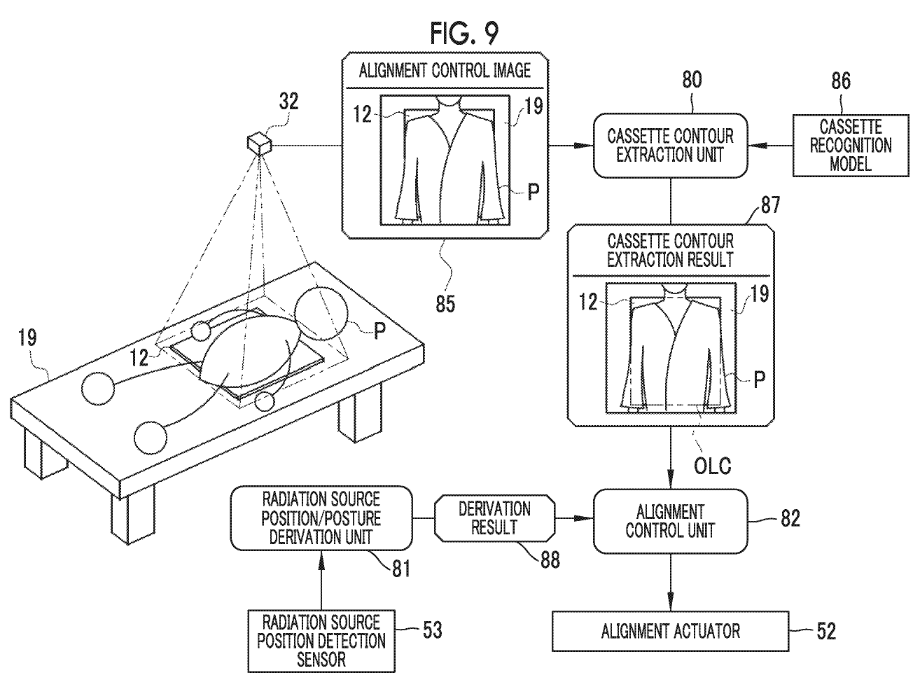

A storage 41 is configured by a non-volatile memory such as a hard disk drive or a solid state drive. The storage 41 stores an operation program 42 and control data 43. The operation program 42 is an example of an "operation program of a radiation generation apparatus" according to the disclosed technology. The control data 43 includes data for driving control, data for alignment control, and data for imaging control. Examples of the data for driving control include map data 73 (see FIG. 8) created in the SLAM method. Examples of the data for alignment control include a cassette recognition model 86 (see FIG. 9) for recognizing the electronic cassette 12. Examples of the data for imaging control include an irradiation condition table in which an irradiation condition for each imaging part is registered.

The operation panel 28, a communication interface (I/F) 50, a driving actuator 51, an alignment actuator 52, and a radiation source position detection sensor 53 are connected to the processor 40. The processor 40 performs display control of various screens on the operation panel 28. In addition, the processor 40 receives various operation instructions of the operator OP through the operation panel 28 and executes various controls corresponding to the various operation instructions. The communication I/F 50 is, for example, a wireless communication I/F and performs wireless communication with the electronic cassette 12.

The driving actuator 51 includes a motor for causing the wheels 15 to drive-rotate and a motor for causing the wheels 15 to revolve, under the control of the processor 40. In addition, the driving actuator 51 also includes a driving state detection sensor 51A that measures a rotation direction and a rotation amount of the wheels 15 and a revolution direction and a revolution angle. The driving state detection sensor 51A is, for example, a rotary encoder or a gyro sensor, or a combination of a plurality of types of these sensors. The driving state detection sensor 51A outputs the measurement value to the processor 40. The processor 40 detects the driving state such as the moving speed and the movement direction, in addition to the movement amount of the carriage unit 16, based on the measurement value from the driving state detection sensor.

The driving state can be used for various purposes. For example, the processor 40 can determine whether or not an operation state of the driving mechanism including the carriage unit 16 is normal based on the driving state detection sensor 51A.

In addition, the processor 40 uses the SLAM method as one of driving control methods for autonomous driving, but it is also possible to execute a driving control method other than the SLAM method by using the driving state detection sensor 51A. As will be described later, the SLAM method executes self-position estimation and creation of the map data based on the surrounding environment information to perform the autonomous driving. As a driving control method other than the SLAM method, for example, there is a method of executing the self-position estimation based on the movement amount detected by the driving state detection sensor 51A without creating the map data, and performing the autonomous driving. Of course, such a driving control method tends to have an increased error as the driving distance is longer as compared with the SLAM method, and thus is not suitable in a case in which the driving distance is long. However, it may be effective in a case in which the driving distance is short or the like, in which the method is used as an auxiliary.

The alignment actuator 52 includes a motor for causing the movable part 25 to be raised and lowered, a motor for causing the arm 27 to be raised and lowered, a motor for bending the second portion of the arm 27, a motor for extending and contracting the second portion, and a motor for rotating the radiation source 13 with respect to the second portion, under the control of the processor 40.

The radiation source position detection sensor 53 measures an elevation direction and an elevation amount of the arm 27 with respect to the movable part 25, a bending direction and a bending amount of the second portion of the arm 27 with respect to the first portion, an extension direction and an extension amount of the second portion, and a rotation direction and a rotation amount of the radiation source 13 with respect to the second portion. The radiation source position detection sensor 53 is, for example, a rotary encoder, a potentiometer, a gyro sensor, or a combination of a plurality of types of these sensors. The radiation source position detection sensor 53 outputs the measurement value to the processor 40. The processor 40 derives the position and the posture of the radiation source 13 based on the measurement value of the radiation source position detection sensor 53.

Since the radiation generation apparatus 11 can autonomously drive, the radiation generation apparatus 11 can automatically move to a designated position. As shown in FIGS. 5 and 6 as an example, a standby position HP of the radiation generation apparatus 11 is prepared in a corner of the imaging room RM. In the standby position HP, the radiation source 13 is in the accommodation position. In the standby position HP, charging of the battery BT, transmission of an imaging order from a radiology information system (RIS), setting of an irradiation condition, and the like are performed. The standby position HP occupies a region that is the same as or one size larger than the radiation generation apparatus 11.

In the imaging room RM, a first target position TP1 (see FIG. 5) of the radiation generation apparatus 11 in the decubitus imaging and a second target position TP2 (see FIG. 6) of the radiation generation apparatus 11 in the upright imaging are set. The first target position TP1 is a position on a side of the decubitus imaging table 19, and more specifically, a position facing a center portion of one long side of the decubitus imaging table 19. The second target position TP2 is a position facing the upright imaging table 20 at a distance of a source-to-image distance (SID) required for the upright imaging. In the following, the first target position TP1 and the second target position TP2 may be collectively referred to as a target position TP.

In a case of the decubitus imaging, the radiation generation apparatus 11 drives, for example, from the standby position HP toward the first target position TP1 and stops in a case in which the self-position PS (see FIG. 8) reaches the first target position TP1 (see FIG. 5). In addition, in a case of the upright imaging, the radiation generation apparatus 11 drives from the standby position HP toward the second target position TP2 and stops in a case in which the self-position PS reaches the second target position TP2. Although not shown, the radiation generation apparatus 11 can also drive from the first target position TP1 toward the second target position TP2 in order to perform the upright imaging after the decubitus imaging. In addition, the radiation generation apparatus 11 can also drive from the second target position TP2 toward the first target position TP1 in order to perform the decubitus imaging after the upright imaging. In a case of the autonomous driving to the target position TP, the radiation source 13 is in the accommodation position.

The autonomous driving to the target position TP is started, for example, by an instruction of the operator OP through the operation panel 28. In addition to or instead of the operation panel 28, a configuration may be adopted in which an instruction to start the autonomous driving can be issued by a remote controller.

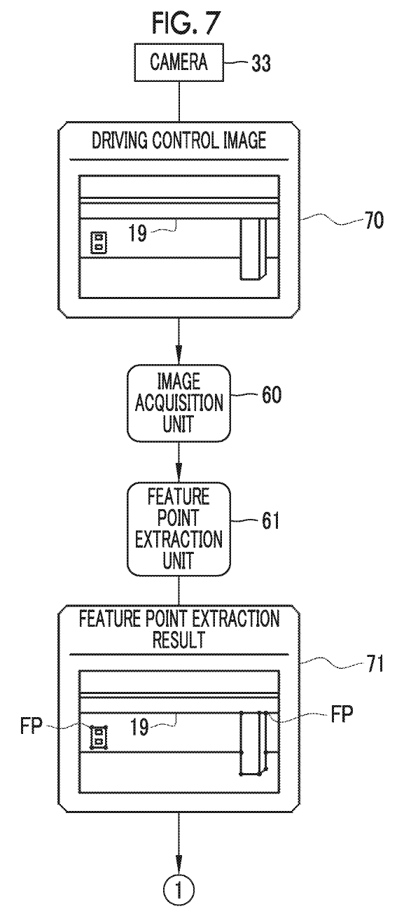

In the SLAM method, the self-position estimation is performed by continuously acquiring an image representing the surrounding environment and tracking the movement of the feature point in the image by image analysis, thereby realizing the autonomous driving. Specifically, as shown in FIGS. 7 and 8, the processor 40 functions as an image acquisition unit 60, a feature point extraction unit 61, a self-position estimation/map data creation unit 62, and a driving control unit 63 by the activation of the operation program 42.

The image acquisition unit 60 sequentially acquires an image 70 (hereinafter, referred to as a driving control image) of the surrounding environment of the radiation generation apparatus 11 captured by the camera 33. A frame rate, which is a frequency of acquiring the driving control image 70, is preset as an operation condition. The image acquisition unit 60 performs preprocessing such as noise removal and distortion correction on the driving control image 70, and then outputs the driving control image 70 to the feature point extraction unit 61.

The feature point extraction unit 61 extracts a corner of a structure present in the surrounding environment shown in the driving control image 70 as a feature point FP by using an algorithm such as oriented features from accelerated segment test and rotated binary robust independent elementary features (ORB) or speeded-up robust features (SURF). The feature point extraction unit 61 outputs a feature point extraction result 71 of the feature point FP to the self-position estimation/map data creation unit 62. In addition, although not shown, the feature point extraction unit 61 stores the feature point extraction result 71 in the storage 41. The feature point extraction result 71 is a set of coordinates and a feature amount vector of each feature point FP.

The self-position estimation/map data creation unit 62 estimates the self-position PS of the radiation generation apparatus 11 and creates the map data 73 of the surrounding environment. The feature point extraction result 71 is input to the self-position estimation/map data creation unit 62 from the feature point extraction unit 61. In addition, a feature point extraction result (hereinafter, referred to as an extraction result (past result)) 71P for a plurality of past frames and map data (hereinafter, referred to as map data (past data)) 73P for a plurality of past frames are input to the self-position estimation/map data creation unit 62.

The feature point extraction result (past result) 71P and the map data (past data) 73P are stored in the storage 41 as the data for driving control of the control data 43. The feature point extraction result 71 and the map data 73 constituting the feature point extraction result (past result) 71P and the map data (past data) 73P are, for example, for several tens to several hundreds of frames. The feature point extraction result (past result) 71P and the map data (past data) 73P include the feature point extraction result 71 and the map data 73 that are considered to play an important role in the estimation of the self-position PS and the creation of the map data 73. The feature point extraction result 71 and the map data 73 are, for example, the feature point extraction result 71 and the map data 73 obtained for each movement of a certain distance. In addition, for example, the feature point extraction result 71 and the map data 73 obtained in a case in which a large viewpoint change has occurred from the previous frame. Further, for example, the feature point extraction result 71 and the map data 73 obtained in a case in which a set amount or more of new feature points FP are extracted.

The self-position estimation/map data creation unit 62 collates the feature point FP of the feature point extraction result 71 from the feature point extraction unit 61 with the feature point FP of the feature point extraction result (past result) 71P. In this case, the self-position estimation/map data creation unit 62 refers to the feature amount vector of each feature point FP. More specifically, the feature point extraction unit 61 recognizes the feature points FP having a distance (Euclidean distance or the like) of the feature amount vector less than the threshold value between the feature point FP of the feature point extraction result 71 from the feature point extraction unit 61 and the feature point FP of the feature point extraction result (past result) 71P as the same feature point FP. The self-position estimation/map data creation unit 62 estimates the self-position PS based on the collation result of the feature point FP and the map data (past data) 73P. In addition, the self-position estimation/map data creation unit 62 creates (updates the map data 73) the new map data 73 based on the feature point extraction result 71 from the feature point extraction unit 61, the feature point extraction result (past result) 71P, the map data (past data) 73P, and an estimation result 72. As described above, the self-position estimation/map data creation unit 62 estimates the self-position PS and creates the map data 73 in parallel in a process in which the radiation generation apparatus 11 autonomously drives.

The self-position estimation/map data creation unit 62 outputs the estimation result 72 of the self-position PS to the driving control unit 63. The driving control unit 63 controls the driving of the driving actuator 51 such that the self-position PS is the target position TP. In addition, although not shown, the self-position estimation/map data creation unit 62 stores the map data 73 in the storage 41. Specifically, the map data 73 is three-dimensional data of the imaging room RM including a structure such as the decubitus imaging table 19 and the upright imaging table 20. In addition, the standby position HP and the target position TP are registered in the map data 73.

As shown in FIG. 9 as an example, the processor 40 functions as a cassette contour extraction unit 80, a radiation source position/posture derivation unit 81, and an alignment control unit 82.

An image 85 (hereinafter, referred to as an alignment control image) including the patient P and the electronic cassette 12 captured by the camera 32 is sequentially input to the cassette contour extraction unit 80. The camera 32 is attached to the radiation source 13. Therefore, in a case in which the self-position PS of the radiation generation apparatus 11 is the target position TP and the radiation source 13 is the imaging preparation position, the patient P and the electronic cassette 12 are shown in the alignment control image 85. In FIG. 9, since the decubitus imaging is shown as an example, the decubitus imaging table 19 is also shown in the alignment control image 85. It is assumed that the alignment between the patient P and the electronic cassette 12 is completed by the operator OP before the alignment control.

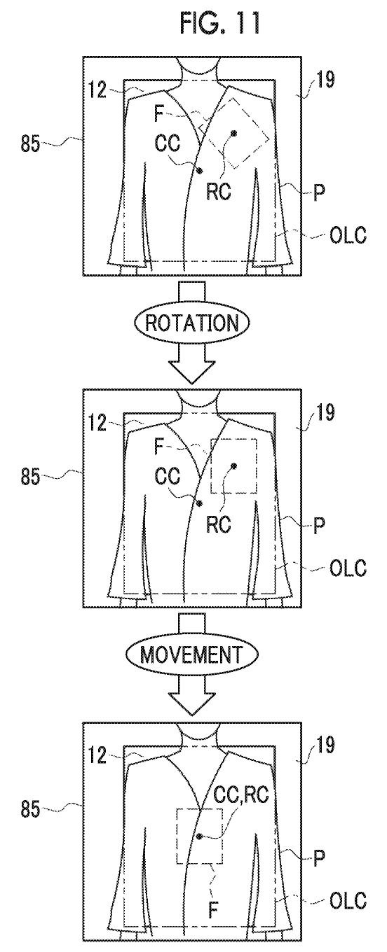

The cassette contour extraction unit 80 extracts a contour OLC of the electronic cassette 12 from the alignment control image 85 by using the cassette recognition model 86. According to the contour OLC, a center CC (see FIG. 11) of the detection surface of the electronic cassette 12 is known. The cassette contour extraction unit 80 outputs a cassette contour extraction result 87 to the alignment control unit 82. Here, the contour following all sides of the electronic cassette 12 is shown as the contour OLC, but the present disclosure is not limited to this. Four corners of the electronic cassette 12 may be extracted as the contour. The alignment control image 85 is an example of "output data" according to the disclosed technology.

The cassette recognition model 86 is a trained model that is configured by, for example, a convolutional neural network or the like and that has been trained to output the cassette contour extraction result 87 in a case in which the alignment control image 85 is input. The cassette recognition model 86 is stored in the storage 41 as the data for alignment control of the control data 43.

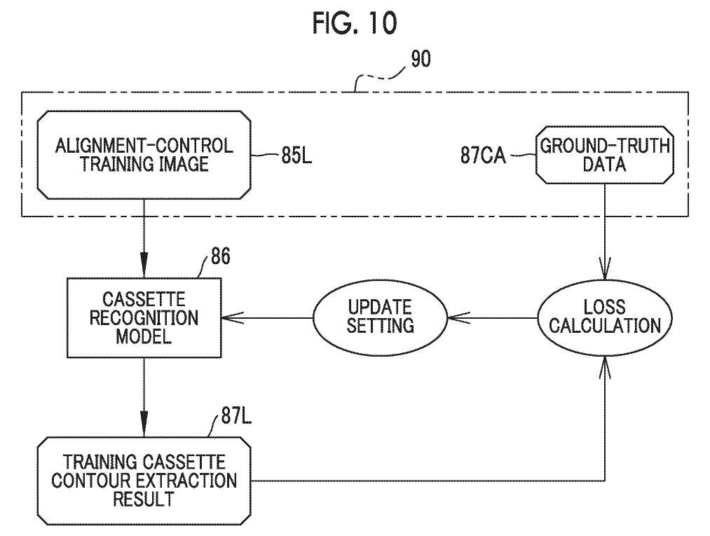

As shown in FIG. 10 as an example, in the learning phase, learning data 90 is given to the cassette recognition model 86. The learning data 90 is composed of a set of an alignment-control training image 85L and ground-truth data 87CA. The electronic cassette 12 is shown in the alignment-control training image 85L. The ground-truth data 87CA is data in which the contour OLC of the electronic cassette 12 shown in the alignment-control training image 85L is annotated, and is data for checking the answer.

The alignment-control training image 85L is input to the cassette recognition model 86. The cassette recognition model 86 outputs a training cassette contour extraction result 87L in response to the input of the alignment-control training image 85L. The loss calculation of the cassette recognition model 86 using the loss function is performed based on the training cassette contour extraction result 87L and the ground-truth data 87CA. Then, the update setting of various coefficients (coefficients of a filter of a convolutional layer and the like) of the cassette recognition model 86 is made according to the result of the loss calculation, and the cassette recognition model 86 is updated according to the update setting.

In the learning phase of the cassette recognition model 86, the series of processing of the input of the alignment-control training image 85L to the cassette recognition model 86, the output of the training cassette contour extraction result 87L from the cassette recognition model 86, the loss calculation, the update setting, and the update of the cassette recognition model 86 is repeatedly performed while the learning data 90 is replaced. The repetition of the series of processing is ended in a case in which the extraction accuracy of the training cassette contour extraction result 87L reaches a predetermined set level. The cassette recognition model 86 in which the extraction accuracy has reached the set level is stored in the storage 41 and is used by the cassette contour extraction unit 80. It should be noted that the learning may be ended in a case in which the series of processing is repeated a set number of times regardless of the extraction accuracy of the training cassette contour extraction result 87L.

The radiation source position/posture derivation unit 81 derives the position and the posture of the radiation source 13 based on the measurement value of the radiation source position detection sensor 53. The radiation source position/posture derivation unit 81 outputs a derivation result 88 to the alignment control unit 82. The derivation result 88 is coordinates of an irradiation center RC (see FIG. 11) of the radiation R in the alignment control image 85 and coordinates of a rectangular frame F (see FIG. 11) indicating the posture of the radiation source 13 in the alignment control image 85. Since the positional relationship between the radiation source 13 and the camera 32 is known, the coordinates of the irradiation center RC and the frame F in the alignment control image 85 can be easily converted from the measurement value of the radiation source position detection sensor 53.

The alignment control unit 82 controls the driving of the alignment actuator 52 such that the radiation source 13 and the electronic cassette 12 face each other. More specifically, as shown in FIG. 11 as an example, in a case in which the posture of the radiation source 13 is inclined with respect to the electronic cassette 12, the alignment control unit 82 rotates the radiation source 13 to eliminate the inclination. In addition, in a case in which the irradiation center RC of the radiation R and the center CC of the detection surface of the electronic cassette 12 are shifted, the alignment control unit 82 moves the radiation source 13 to eliminate the shift. It should be noted that the inclination may be eliminated after the shift between the irradiation center RC of the radiation R and the center CC of the detection surface of the electronic cassette 12 is eliminated. In addition, in FIG. 11, a case in which the posture of the radiation source 13 is inclined around a normal line of the detection surface of the electronic cassette 12 is shown as an example, but the present disclosure is not limited to this. Even in a case in which the posture of the radiation source 13 is inclined around an axis along the long side of the detection surface of the electronic cassette 12 or around an axis along the short side of the detection surface of the electronic cassette 12, the alignment control unit 82 rotates the radiation source 13 to eliminate the inclination.

As shown in FIG. 12, the alignment control unit 82 displays an alignment support screen 91 showing a state of the alignment between the radiation source 13 and the electronic cassette 12 on the operation panel 28. The alignment control image 85 is displayed on the alignment support screen 91. The operator OP can check the state of the alignment between the radiation source 13 and the electronic cassette 12 through the alignment support screen 91.

In addition, although the example in which the alignment of the radiation source 13 is automatically performed by the alignment control unit 82 has been described, the operator OP may manually perform the alignment of the radiation source 13. The manual operation may be, for example, an operation in which the operator OP inputs an operation instruction to the alignment actuator 52 through an operation button or the like, or an operation in which the operator OP directly moves the radiation source 13. Of course, in a case in which the radiation source 13 is displaced by the manual operation of the operator OP, the alignment control image 85 in the alignment support screen 91 is updated. As a result, the operator OP can check the current state of the alignment through the alignment support screen 91.

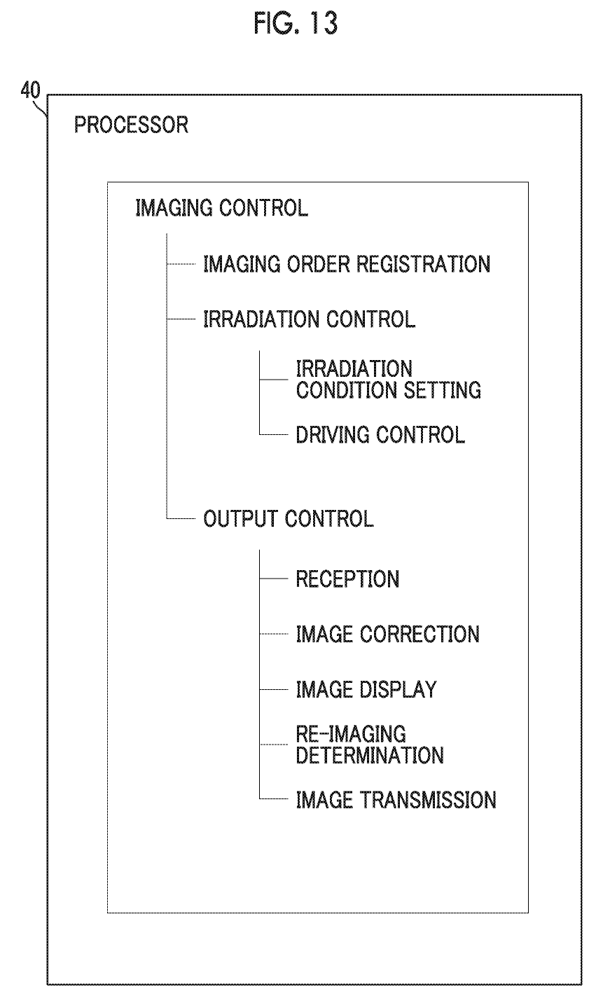

As shown in FIG. 13, the processor 40 further executes the imaging control. The imaging control includes, for example, control such as imaging order registration, irradiation control, and output control. The imaging order registration is processing of registering information such as patient information, an imaging purpose, and an imaging part based on an imaging order received from the RIS or the like.

The irradiation control includes irradiation condition setting and driving control. The irradiation condition setting is setting of an irradiation condition including a tube voltage, a tube current, and an irradiation time of the radiation generated by the radiation source 13. The driving control is driving control of the radiation source 13 according to the set irradiation condition, and the driving control includes synchronization control of an irradiation timing of the radiation source 13 and an image detection timing of the electronic cassette 12.

The output control includes processing of receiving the radiographic image 18 detected by the electronic cassette 12, image correction of performing various types of correction such as offset correction, sensitivity correction, and defect correction on the received radiographic image 18, image display of displaying the corrected radiographic image 18 on the operation panel 28, and the like. Further, the output control includes re-imaging determination and image transmission. The re-imaging determination is processing of determining whether or not the imaged radiographic image 18 can be used for diagnosis in light of the imaging purpose, and determining whether or not re-imaging is necessary. The image transmission is processing of transmitting the imaged radiographic image 18 to an image server.

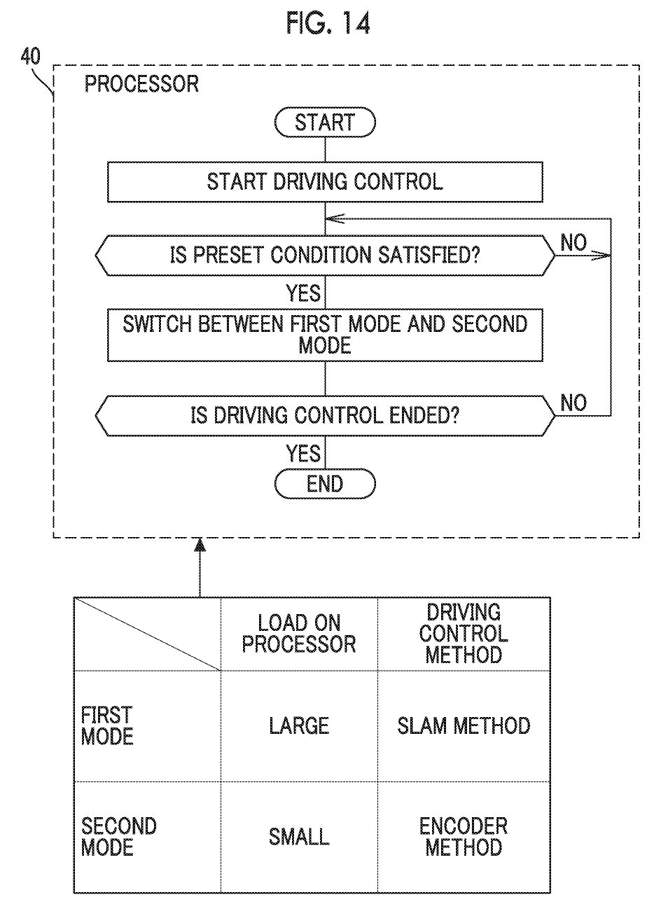

Further, as shown in FIG. 14, the processor 40 can switch between the first mode in which a load on the processor 40 is relatively large and the second mode in which the load is relatively small, according to a preset condition, with respect to the mode in which the driving control is executed. The driving control method of the first mode is the SLAM method. The driving control method of the second mode is a driving control method other than the above-described SLAM method, and is, for example, a mode in which the movement amount is detected by the driving state detection sensor 51A instead of acquiring the surrounding environment information, the self-position estimation is executed based on the detected movement amount, and the autonomous driving is performed toward the preset target position TP. Since the driving state detection sensor 51A includes an encoder, the driving control method of the second mode is hereinafter referred to as an encoder method for convenience. It is considered that the encoder method has a larger error than the SLAM method, but the self-position estimation can be performed by accumulating the movement amount, and the autonomous driving can be performed based on the self-position estimated in this way.

The SLAM method of the first mode repeats the image processing of performing the self-position estimation and creating the map data 73 based on the driving control image 70, which is the surrounding environment information, and thus the load on the processor 40 is large. On the other hand, the encoder method of the second mode executes the self-position estimation based on the movement amount detected by the driving state detection sensor 51A, and thus the load on the processor 40 is smaller than that in the first mode.

As shown in FIGS. 9 to 13, the alignment control and the imaging control are executed in addition to the driving control. Therefore, in a case in which these controls need to be performed in parallel, the load on the processor 40 is extremely large. Therefore, as shown in FIG. 14, in a case in which the preset condition is satisfied, the driving control method is switched from the first mode to the second mode. For example, in a case in which the alignment control and the driving control need to be performed in parallel, the mode of the driving control is switched from the first mode to the second mode. Then, in a case in which the other control is ended, the processor 40 returns to the first mode.

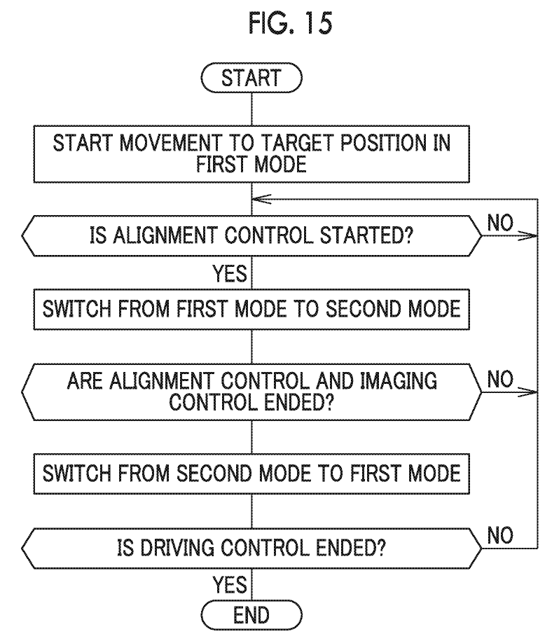

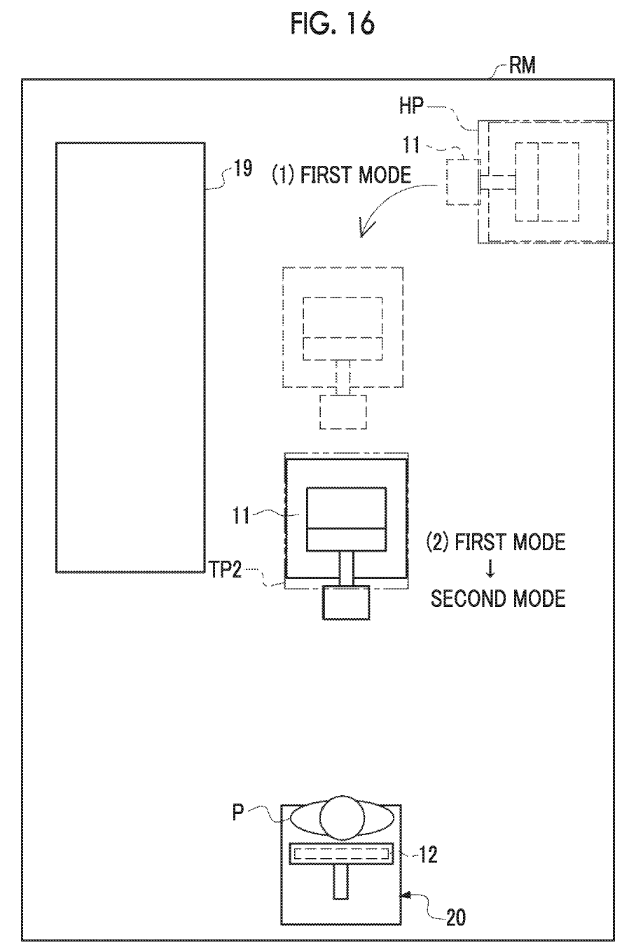

FIGS. 15 and 16 show an example of the mode switching. In a case in which the radiation generation apparatus 11 moves from the standby position HP to the target position TP, the radiation generation apparatus 11 starts the movement to the target position TP in the first mode.

Then, during the movement to the target position TP, the alignment control of the radiation source 13 and the electronic cassette 12 as shown in FIGS. 9 to 12 may be started. In this case, the alignment control and the driving control are performed in parallel. The alignment control is also processing including the image processing, and the load on the processor 40 is large. Therefore, the processor 40 switches the mode of the driving control from the first mode to the second mode having a smaller load. As a result, since a large part of the resources of the processor 40 can be allocated to the alignment control, it is unlikely that a problem occurs in which the alignment control takes a long time or takes a long time.

In addition, as described above, it is considered that the encoder method of the second mode has an increased error as the movement amount is larger than the SLAM method of the first mode, but the error is small in a case in which the movement amount is small. Therefore, it is effective as the driving control method of performing the fine adjustment in the vicinity of the target position TP.

In the radiography workflow, in a case in which the alignment control is ended, the radiography is performed, and the processor 40 executes the imaging control. Then, as shown in FIG. 15, in a case in which the alignment control and the imaging control are ended, the processor 40 switches the mode of the driving control from the second mode to the first mode. Then, for example, the radiation generation apparatus 11 returns to the standby position HP in the first mode. In a case of performing the next imaging, the driving control is started in the first mode, and the above-described processing is repeated.

As described above, the radiation generation apparatus 11, which is an example of the radiography apparatus according to the disclosed technology, comprises the processor 40 that executes the driving control related to the driving mechanism including the carriage unit 16 as an example, and the processor 40 switches between the first mode in which the load on the processor 40 is relatively large and the second mode in which the load is relatively small, according to the preset condition with respect to the mode in which the driving control is executed. As a result, it is possible to appropriately allocate the resources of the processor 40.

In the above-described embodiment, the processor 40 further executes the alignment control that supports the relative alignment between the radiation source 13 and the electronic cassette 12 (an example of the radiographic image detection device). As shown in FIG. 15, the preset condition is the start of the alignment control, and the processor 40 executes the driving control in the first mode before the start of the alignment control and executes the driving control in the second mode in a case in which the alignment control is started. The alignment control may have a large load on the processor 40. Therefore, by reducing the load of the driving control, the resources of the processor 40 can be allocated to the alignment control.

In the above-described embodiment, the first mode is a mode in which the autonomous driving is performed toward the preset target position by executing the self-position estimation and the creation of the map data 73 based on the surrounding environment information as in the SLAM method. The second mode is a mode in which the autonomous driving is performed toward the preset target position by detecting the movement amount of the driving mechanism without creating the map data 73. The first mode such as the SLAM method has a large load on the processor 40, whereas the second mode such as the encoder method has a small load on the processor 40. Therefore, in the processor 40, a high load reduction effect by the switching to the second mode can be expected.

In the above-described embodiment, the preset condition may be the reaching of the target position TP. Even after reaching the target position TP, a new target position may be set to perform the fine adjustment, and the autonomous driving may be performed. Therefore, the processor 40 executes the driving control in the first mode until the first set target position TP is reached, and then executes the driving control in the second mode in a case in which the autonomous driving is further performed. In a case in which the first set target position TP is reached, the probability that the alignment control is started is high, and thus there is an advantage in performing such control in order to allocate the resources of the processor 40 to the alignment control.

In the above-described embodiment, the alignment control that supports the relative alignment between the radiation source 13 and the electronic cassette 12 (an example of the radiographic image detection device) has been described as an example of the alignment control. However, the alignment control may include control of supporting the relative alignment between the radiation source 13 and the patient P (an example of the subject) in addition to the relative alignment between the radiation source 13 and the electronic cassette 12. Further, the alignment target may be the radiation source 13 and the patient P without the electronic cassette 12 as the alignment target. That is, the alignment control means control of supporting the relative alignment between the radiation source 13 and the radiographic image detection device and/or the subject. In addition, the alignment between the radiation source 13 and the patient P also includes alignment between the radiation source 13 and the imaging part (chest, abdomen, head, and limbs) of the patient P.

### Modification Example 1

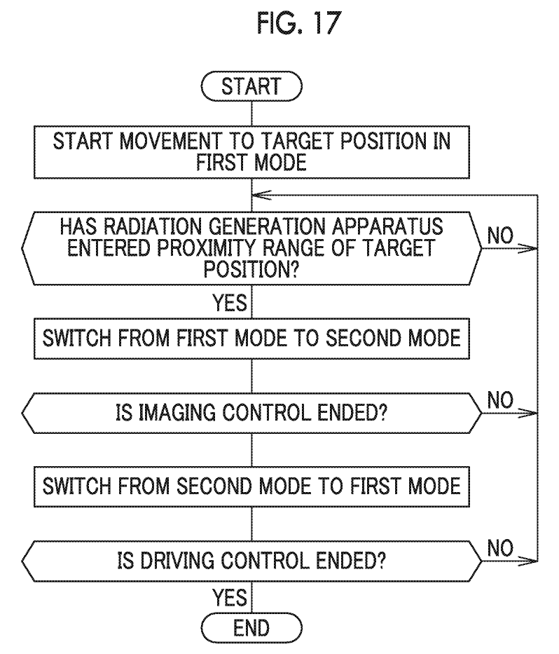

As shown in FIG. 17, the preset condition for switching from the first mode to the second mode is not limited to the start of the alignment control. In the example shown in FIG. 17, it is assumed that the radiation generation apparatus 11 has entered the proximity range of the target position TP as the preset condition. The proximity range is a range preset around the target position TP, and is, for example, a range in which a difference between the target position TP and the self-position is within about 300 mm. The processor 40 executes the driving control in the first mode before entering the proximity range and executes the driving control in the second mode after entering the proximity range. In the radiography workflow, the probability that the alignment control and the imaging control are started at the target position TP is high. Therefore, in the example shown in FIG. 17, in a case in which the radiation generation apparatus 11 has entered the proximity range of the target position TP in anticipation of the start of the alignment control and the imaging control, the mode is switched to the second mode. The other aspects of the example shown in FIG. 17 are the same as those of the example shown in FIG. 15. Even in the example shown in FIG. 17, the effect of being able to allocate the resources of the processor 40 to the alignment control and the imaging control by reducing the load of the driving control can be obtained.

### Modification Example 2

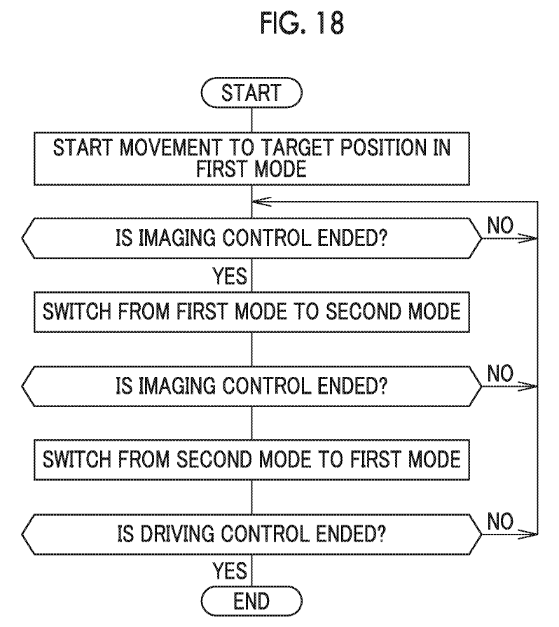

The example shown in FIG. 18 is an example in which the start of the imaging control is set as the preset condition for switching from the first mode to the second mode. That is, in the example shown in FIG. 18, in a case in which a part of the imaging control is started, the mode of the driving control is switched to the second mode. As shown in FIG. 13, the imaging control also includes processing such as the imaging order registration, and the imaging order registration and the driving control may be performed in parallel. In a case in which the imaging order registration is performed, the probability that the alignment control and other imaging controls are executed is high. Therefore, in the example shown in FIG. 18, in a case in which a part of the imaging control is started in anticipation of the start of the alignment control or the like, the mode is switched to the second mode, as in the example shown in FIG. 17. The other aspects of the example shown in FIG. 18 are the same as those of the example shown in FIG. 17. Even in the example shown in FIG. 18, the effect of being able to allocate the resources of the processor 40 to the alignment control and the imaging control by reducing the load of the driving control can be obtained.

### Modification Example 3

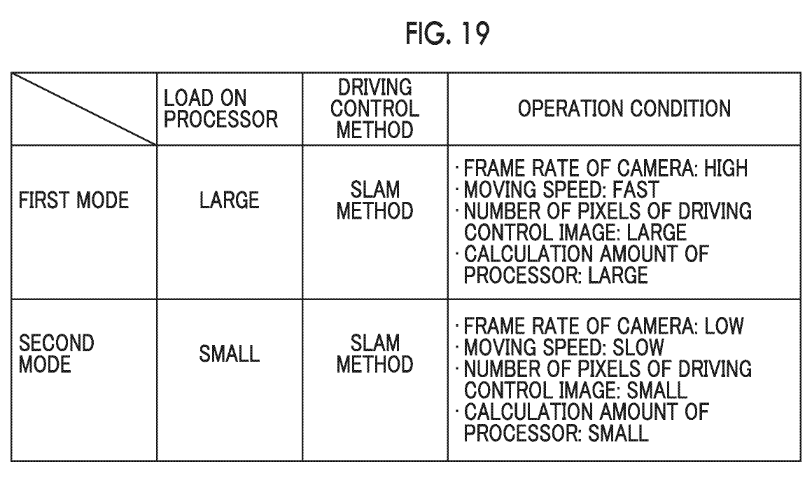

In the above-described embodiment, the second mode has been described as the encoder method, but the present disclosure is not limited to this. As in the example shown in FIG. 19, the second mode may also be the same SLAM method as the first mode. In this case, the first mode and the second mode have different operation conditions such that the load of the second mode is lower than that of the first mode. As shown in FIG. 19, the operation condition includes at least one of a frame rate of the camera 33 (an example of the acquisition frequency of the surrounding environment information), a moving speed of the driving mechanism, the number of pixels of the driving control image 70 (an example of the information amount of the environment information), or a calculation amount of the processor 40. The number of pixels may be changed, for example, by changing the resolution at the time of capturing the driving control image 70 or by thinning out the captured image. The calculation amount of the processor 40 is, for example, the frequency of the self-position estimation and the update of the map data in the SLAM method. It goes without saying that the calculation amount of the processor 40 is also reduced as the number of pixels is reduced. Each item of the operation condition is not exclusive, and may be related to each other.

The second mode satisfies at least one condition of a condition in which the frame rate is low, the moving speed is slow, the number of pixels of the driving control image 70 is small, or the calculation amount is small, as compared with the first mode. As a result, the second mode has a smaller load on the processor 40 than the first mode. Even in a case in which such a second mode is used, it is possible to appropriately allocate the resources of the processor 40 as in the above-described embodiment.

Hereinafter, specific numerical examples of the operation conditions of the first mode and the second mode will be described. The frame rate of the first mode is, for example, 4 frames per second (FPS), and the moving speed is 400 [mm/sec]. The moving speed in a case in which the radiation generation apparatus 11 is rotationally moved is 10 [degrees/sec]. The moving speed of 400 [mm/sec] is set as a speed at which, for example, a movement distance of 8 m can be moved within 20 sec, assuming an imaging room RM of about 3 m × 5 m. On the other hand, the frame rate of the second mode is 2 to 3 FPS. In addition, the moving speed of the second mode is 100 to 150 [mm/sec], and the moving speed in a case of rotation is 5 [degrees/sec]. This is set as a speed at which, for example, the fine adjustment range of the radiation generation apparatus 11 is about 300 [mm/sec] and can be completed within 3 seconds.

### Modification Example 4

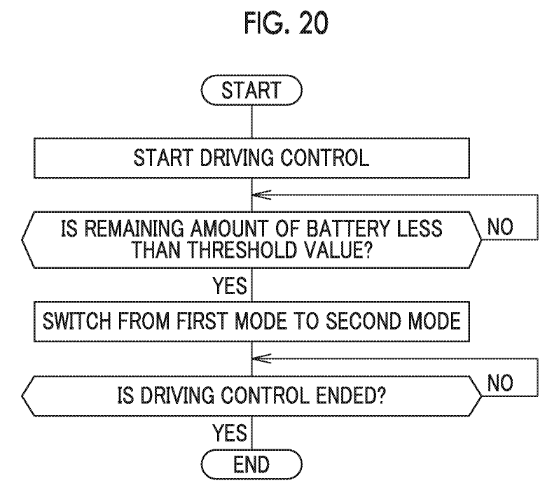

In addition, since the second mode has a smaller load on the processor 40 than the first mode, it is considered that the consumption amount of the battery BT is also small. The example shown in FIG. 20 is an example of suppressing the consumption in a case in which the remaining amount of the battery BT is small. In the example shown in FIG. 20, the preset condition is set as a case in which the remaining amount of the battery BT has decreased to be less than a preset threshold value, and the processor 40 sets the first mode in a case in which the remaining amount is equal to or larger than the threshold value and switches to the second mode in a case in which the remaining amount has decreased to be less than the threshold value. As a result, the consumption of the battery BT can be suppressed in a case in which the remaining amount of the battery BT is decreased. The switching between the first mode and the second mode can also be used for such a purpose.

In the example shown in FIG. 17, the operation condition may be changed stepwise according to the difference between the target position TP and the self-position. For example, in a case in which the difference between the target position TP and the self-position is within a range of about 300 mm, the frame rate of the camera 33 is reduced from 4 FPS to 3 FPS, and then in a case in which the difference reaches about 10 mm, the frame rate is reduced from 3 FPS to 1 to 2 FPS.

In addition, the above-described embodiment is an example, and can be appropriately changed as follows.

The carriage unit 16 may be omitted, and the wheels 15 may be directly attached to a lower portion of the body part 14.

The camera 32 may be attached to the arm 27 instead of the radiation source 13. Similarly, the camera 33 may be provided in the body part 14 instead of the carriage unit 16. A plurality of the cameras 32 and 33 may be provided.

The camera 32 and the camera 33 may be integrated into one camera. In this case, the orientation of the camera is set to an orientation in which the driving control image 70 can be captured at the accommodation position, and an orientation in which the alignment control image 85 can be captured at the imaging preparation position.

The environment information sensor is not limited to the example of the camera 33. A light detection and ranging (LiDAR) sensor, a time-of-flight (TOF) sensor, or the like may be used. In addition, an inertial measurement unit (IMU) in which an acceleration sensor and a gyro sensor are combined, an ultrasonic sensor, a radar sensor, a magnetic sensor, or the like may be used.

In FIG. 19, the frame rate of the camera 33 is shown as the operation condition of the first mode and the second mode, but the operation condition in a case of the camera 33 other than the camera 33 is also considered. For example, the information acquisition frequency corresponding to the type of the environment information sensor, such as the acquisition frequency of the distance image in a case of LiDAR, is included.

In addition, the radiographic image detection device is not limited to the example of the electronic cassette 12. A computed radiography (CR) cassette may be used. In addition, the radiographic image detection device may be an apparatus in which the radiographic image detector is fixed to an imaging table. In addition, the subject is not limited to the example of the patient P. The subject may be a diseased animal such as a dog or a cat.

In addition, the radiation generation apparatus 11 has been described as the radiography apparatus, but the radiography apparatus may be a radiographic image detection device including a driving mechanism.

The above description discloses the following Supplementary notes.

### Supplementary Note 1

A radiography apparatus which is used for radiography, the radiography apparatus comprising:

a radiation source or a radiographic image detection device;

a driving mechanism that is autonomously drivable; and

a processor configured to execute driving control related to the driving mechanism,

wherein the processor switches between a first mode in which a load on the processor is relatively large and a second mode in which the load is relatively small, according to a preset condition, with respect to a mode in which the driving control is executed.

### Supplementary Note 2

The radiography apparatus according to Supplementary Note 1,

wherein the processor is configured to further execute alignment control that supports relative alignment between the radiation source and the radiographic image detection device and/or a subject, and imaging control related to the radiation source or the radiographic image detection device,

the preset condition includes start of at least one of the alignment control or the imaging control, and

the processor is configured to execute the driving control in the first mode before the start of at least one of the controls and execute the driving control in the second mode in a case of starting at least one of the controls.

### Supplementary Note 3

The radiography apparatus according to Supplementary Note 1 or 2,

wherein the first mode is a mode in which autonomous driving is performed toward a preset target position by executing self-position estimation based on surrounding environment information, and

the second mode is a mode in which autonomous driving is performed toward a preset target position by detecting a movement amount of the driving mechanism and executing self-position estimation based on the detected movement amount.

### Supplementary Note 4

The radiography apparatus according to any one of Supplementary Notes 1 to 3,

wherein the first mode and the second mode are modes in which autonomous driving is performed toward a preset target position by executing self-position estimation based on surrounding environment information, and

the first mode and the second mode have different operation conditions.

### Supplementary Note 5

The radiography apparatus according to Supplementary Note 4,

wherein the operation condition includes at least one of an acquisition frequency of the surrounding environment information, a moving speed of the driving mechanism, an information amount of the environment information, or a calculation amount of the processor, and

the second mode is a mode that satisfies at least one of a condition in which the acquisition frequency is low, a condition in which the moving speed is slow, a condition in which the information amount of the environment information is small, or a condition in which the calculation amount is small, as compared with the first mode.

### Supplementary Note 6

The radiography apparatus according to any one of Supplementary Notes 1 to 5,

wherein the first mode is a mode in which autonomous driving is performed toward a preset target position by executing self-position estimation based on surrounding environment information,

the preset condition includes that the self-position has entered a preset proximity range of the target position, and

the processor is configured to execute the driving control in the first mode before entering the proximity range and execute the driving control in the second mode after entering the proximity range.

### Supplementary Note 7

The radiography apparatus according to any one of Supplementary Notes 1 to 6,

wherein the radiography apparatus is driven by a battery.

### Supplementary Note 8

The radiography apparatus according to Supplementary Note 7,

wherein the preset condition includes that a remaining amount of the battery has decreased to be less than a preset threshold value, and

the processor is configured to execute the driving control in the first mode in a case where the remaining amount is equal to or larger than the threshold value and execute the driving control in the second mode in a case where the remaining amount has decreased to be less than the threshold value.

### Supplementary Note 9

The radiography apparatus according to any one of Supplementary Notes 1 to 8,

wherein the radiography apparatus includes the radiation source and functions as a radiation generation apparatus.

### Supplementary Note 10

An operation method of a radiography apparatus, in which the radiography apparatus includes a radiation source or a radiographic image detection device, a driving mechanism that is autonomously drivable, and a processor configured to execute driving control related to the driving mechanism, the operation method comprising:

switching, by the processor, between a first mode in which a load on the processor is relatively large and a second mode in which the load is relatively small, according to a preset condition, with respect to a mode in which the driving control is executed.

### Supplementary Note 11

An operation program of a radiography apparatus, in which the radiography apparatus includes a radiation source or a radiographic image detection device, a driving mechanism that is autonomously drivable, and a processor configured to execute driving control related to the driving mechanism, the operation program causing the processor to execute a process comprising:

switching between a first mode in which a load on the processor is relatively large and a second mode in which the load is relatively small, according to a preset condition, with respect to a mode in which the driving control is executed.

In the above-described embodiment, the processing executed by the processor 40 is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate.

Any computer may be a general-purpose computer, a computer for specific use, a workstation, or another system capable of executing each processing. The processor may be configured using one or more pieces of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing, such as an application specific integrated circuit (ASIC), or hardware such as a graphic processing unit (GPU) or a neural processing unit (NPU). Furthermore, the types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is configured by an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium or other storage). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The technology of the present disclosure can also be combined with various embodiments and/or various modification examples described above, as appropriate. In addition, the present disclosure is not limited to the above-described embodiments, and various configurations can be adopted without departing from the gist of the present disclosure. Further, the technology of the present disclosure includes a storage medium that stores the program in a non-transitory manner, in addition to the program. The storage medium is, for example, a non-transitory computer-readable storage medium such as a universal serial bus (USB) memory, a flexible disk, or a compact disc read only memory (CD-ROM). The program may be provided online through a network such as the Internet. The disclosed technology also applies to a program product in addition to the program. The program product includes products of every aspect for providing the program. Like the program, the program product may be provided by being stored in a non-transitory computer-readable storage medium or may be provided online.

The above descriptions and illustrations are detailed descriptions of portions related to the technology of the present disclosure and are merely examples of the technology of the present disclosure. For example, the above description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in the above descriptions and illustrations, the description of, for example, common technical knowledge that does not need to be particularly described to enable the implementation of the technology of the present disclosure is omitted in order to avoid confusion and facilitate the understanding of portions related to the technology of the present disclosure.

In the specification, "A and/or B" is synonymous with "at least one of A or B". That is, "A and/or B" means that it may be only A, only B, or a combination of A and B. Further, in the specification, the same concept as "A and/or B" is applied to a case in which the connection of three or more matters is expressed by "and/or".

All of the documents, the patent applications, and the technical standards described in the specification are incorporated by reference herein to the same extent as each document, each patent application, and each technical standard are specifically and individually stated to be incorporated by reference.

What is claimed is:

1. A radiography apparatus which is used for radiography, the radiography apparatus comprising:
a radiation source or a radiographic image detection device;
a driving mechanism that is autonomously drivable; and
a processor configured to execute driving control related to the driving mechanism,
wherein the processor switches between a first mode in which a load on the processor is relatively large and a second mode in which the load is relatively small, according to a preset condition, with respect to a mode in which the driving control is executed.

2. The radiography apparatus according to claim 1,
wherein the processor is configured to further execute alignment control that supports relative alignment between the radiation source and the radiographic image detection device and/or a subject, and imaging control related to the radiation source or the radiographic image detection device,
the preset condition includes start of at least one of the alignment control or the imaging control, and
the processor is configured to execute the driving control in the first mode before the start of at least one of the controls and execute the driving control in the second mode in a case of starting at least one of the controls.

3. The radiography apparatus according to claim 1,
wherein the first mode is a mode in which autonomous driving is performed toward a preset target position by executing self-position estimation based on surrounding environment information, and
the second mode is a mode in which autonomous driving is performed toward a preset target position by detecting a movement amount of the driving mechanism and executing self-position estimation based on the detected movement amount.

4. The radiography apparatus according to claim 1,
wherein the first mode and the second mode are modes in which autonomous driving is performed toward a preset target position by executing self-position estimation based on surrounding environment information, and
the first mode and the second mode have different operation conditions.

5. The radiography apparatus according to claim 4,
wherein the operation condition includes at least one of an acquisition frequency of the surrounding environment information, a moving speed of the driving mechanism, an information amount of the environment information, or a calculation amount of the processor, and
the second mode is a mode that satisfies at least one of a condition in which the acquisition frequency is low, a condition in which the moving speed is slow, a condition in which the information amount of the environment information is small, or a condition in which the calculation amount is small, as compared with the first mode.

6. The radiography apparatus according to claim 1,
wherein the first mode is a mode in which autonomous driving is performed toward a preset target position by executing self-position estimation based on surrounding environment information,
the preset condition includes that the self-position has entered a preset proximity range of the target position, and
the processor is configured to execute the driving control in the first mode before entering the proximity range and execute the driving control in the second mode after entering the proximity range.

7. The radiography apparatus according to claim 1,
wherein the radiography apparatus is driven by a battery.

8. The radiography apparatus according to claim 7,
wherein the preset condition includes that a remaining amount of the battery has decreased to be less than a preset threshold value, and
the processor is configured to execute the driving control in the first mode in a case where the remaining amount is equal to or larger than the threshold value and execute the driving control in the second mode in a case where the remaining amount has decreased to be less than the threshold value.

9. The radiography apparatus according to claim 1,
wherein the radiography apparatus includes the radiation source and functions as a radiation generation apparatus.

10. An operation method of a radiography apparatus which is used for radiography, and which includes a radiation source or a radiographic image detection device, a driving mechanism that is autonomously drivable, and a processor configured to execute driving control related to the driving mechanism, the operation method comprising:
switching, by the processor, between a first mode in which a load on the processor is relatively large and a second mode in which the load is relatively small, according to a preset condition, with respect to a mode in which the driving control is executed.

11. A non-transitory computer-readable storage medium storing an operation program of a radiography apparatus which is used for radiography, and which includes a radiation source or a radiographic image detection device, a driving mechanism that is autonomously drivable, and a processor configured to execute driving control related to the driving mechanism, the operation program causing the processor to execute a process comprising:
switching between a first mode in which a load on the processor is relatively large and a second mode in which the load is relatively small, according to a preset condition, with respect to a mode in which the driving control is executed.
